(19) United States
(12) Patent Application Publication
(10) Pub. No.: US 2008/0051912 A1
(43) Pub. Date: Feb. 28, 2008

(54) METATARSAL IMPLANT

(75) Inventor: Shane M. Hollawell, Wall Township, NJ (US)

Correspondence Address: MORGAN, LEWIS & BOCKIUS LLP, 1701 MARKET STREET, PHILADELPHIA, PA, 19103-2921, US

(73) Assignee: Small Bone Innovations, Inc.

(21) Appl. No.: 11/744,976

(22) Filed: May 7, 2007

Related U.S. Application Data
(63) Continuation of application No. PCT/US05/40260, filed on Nov. 8, 2005.
(60) Provisional application No. 60/625,987, filed on Nov. 8, 2004.

(51) Int. Cl. A61F 2/66; A61B 17/58

(52) U.S. Cl. 623/54; 606/72

(57) ABSTRACT

The present invention comprises a metatarsal head implant comprising a generally convex, hollow portion that fits over the metatarsal head of the great toe, a serrated stem, and one or more locking screws. The implant of the present invention may work in conjunction with either the patient's existing phalanx or one-piece phalanx implants designed and/or manufactured independently from the metatarsal implant.

### CROSS-RERFERENCE TO RELATED APPLICATIONS

This application claims priority to U.S. provisional application Ser. No. 60/625,987 filed on Nov. 8, 2004 incorporated herein by reference in its entirety.

### FIELD OF THE INVENTION

The present invention relates to devices and methods for the repair of defects and/or diseases of the first metatarsal joint.

### BACKGROUND OF THE INVENTION

The majority of disease seen in the great toe joint affects the head of the first metatarsal. The design of an implant to replace part of the head of the metatarsal must take into account the function of the sesamoid apparatus plantarly as well as the ability to decompress the joint. The shape of the articular component of the head of the metatarsal replacing the actual portion of the head itself must have a specific convex design to articulate with the base of the proximal phalanax, and the implant's design must allow for ball-and-socket type motion to continue. Furthermore, an avoidance of sesamoid impingement syndrome must be employed so that patients do not develop inferior sesamoiditis. Because a large portion of the joints demonstrate disease on tie head of the metatarsal, the design of a joint implant must deal with the diseased portion of the joint and be able to withstand the weight-bearing forces transmitted through the first metatarsophalangeal joint.

Numerous attempts to replace the head of the metatarsal or its articulating surface have been employed with various types of implants, but the vast majority of those efforts have failed. In the past, reconstruction of the first metatarsophalangeal joint has been achieved by either replacing only the phalanx head or replacing both the metatarsal and the phalangeal head with suitable implants. The component joints replacing the base of the proximal phalanx as well as the head of the metatarsal have been subject to extreme failure due to limited motion, implant failure, loosening and displacement of the implant, plantar pain, and disruption of the sesamoid apparatus. Implantation of these implants typically requires the removal of healthy joint tissue; the base of the proximal phalanx is often resected and replaced as part of the hemi implantation to decompress the joint and provide a material, other than hyaline cartilage, that can articulate with the head of the metatarsal with degenerative changes.

Prior art implants have typically used serrations, grooves, or some similar method of preventing slippage of the implant. Such prior methods are either ineffective at preventing slippage of the implant or require a protected weight-bearing arrangement (i.e., a traditional cast, walking cast, or other type of external stabilization) for a period of time before the patient can place their full weight on the implant site.

U.S. Pat. No. 5,458,648 to Berman et al. describes a two-component implant for the replacement of metatarsophalangeal joints. These implants require prosthetic implantation into both the metatarsal and phalangeal bones. Further, implantation therein described requires extensive resection of both the metatarsal and phalangeal bones. Grooves on the surface of the implant stem are the only means by which the implant becomes secured.

U.S. Pat. No. 6,679,917 to Ek describes a one component implant for repairing defects in articular cartilage. These implants do not maintain the natural shape of the metatarsal bone and implantation requires extensive reaming. Threads on the stem of the implant are the only mechanical means by which the implant becomes secured.

### SUMMARY OF THE INVENTION

The present invention comprises a metatarsal head implant for use in the reconstruction of damaged or diseased metatarsal joints of the great toe. In one embodiment of the invention, the implant has a one-piece construction, comprising a generally convex, hollow portion that fits over the metatarsal head of the great toe, a serrated stem, and one or more locking screws. The implant of the present invention is designed to conform to the patient's existing (natural) phalanx, thus eliminating the need for a phalanx implant that is specifically desired to work in conjunction with the metatarsal implant. To reach this goal, the design of the present invention is such that the shape of the implant is more convex than that of previous metatarsal implants.

In a further embodiment of the present invention, the metatarsal implant is designed so that it may work in conjunction with either the patient's existing phalanx or one-piece phalanx implants designed and/or manufactured independently from the metatarsal implant. An additional advantage of the one-piece design of the present invention is the ability to maintain the health and integrity of the existing sesamoid bones, which has previously been difficult when using two-piece metatarsophalangeal implants. In further embodiments of the present invention, the implant may be adapted for use in the hand as well as for other joints of the foot.

Locking screws have previously been used in the orthopedic profession, but not in conjunction with implants that allow a range of motion, i.e., implants of the foot or hand. In the present invention, one or more locking screws are employed to prevent rotation and backing-out of the implant and provide stability in more than one plane. The use of a serrated stem in conjunction with one or more locking screws, as in the present invention, allows immediate and full weight-bearing on the affected joint.

In a further embodiment, a hollow stem having preferred fenestrations is used. The hollow stem is preferably filled with a material which supports, stimulates, or facilitates bone ingrowth through the stem.

### BRIEF DESCRIPTION OF THE DRAWINGS

FIG. 1A is a perspective, posterior side elevational view showing the metatarsal implant of the present invention;

FIG. 1B is a perspective, side elevational view showing the metatarsal implant of the present invention;

FIG. 1C is a perspective, anterior side elevational view showing the metatarsal implant of the present invention;

FIG. 2A is a perspective, plantar side elevational view showing the metatarsal implant of the present invention;

FIG. 2B is a perspective, dorsal side elevational view of the metatarsal implant of the present invention;

FIG. 3A is a detailed view showing a hollow stem of the present invention, having fenestrations;

FIG. 3B is a detailed view showing a hollow stem of the present invention, having apertures;

FIG. 3C is a detailed, cross-sectional view or a hollow stem of the present invention, filled with osteogenic or osteostimulatory material;

FIG. 4A is a side elevational view illustrating the implantation of the implant of the present invention into the first metatarsal bone;

FIG. 4B is a side elevational view, illustrating the full range of motion of the metatarsophalangeal joint, after implantation of the implant of the present invention;

FIG. 4C is a cross-sectional view of the first metatarsal bone, showing the implantation of the implant of the present invention, including fasteners securing the implant to the bone.

### DETAILED DESCRIPTION OF ILLUSTRATIVE EMBODIMENTS

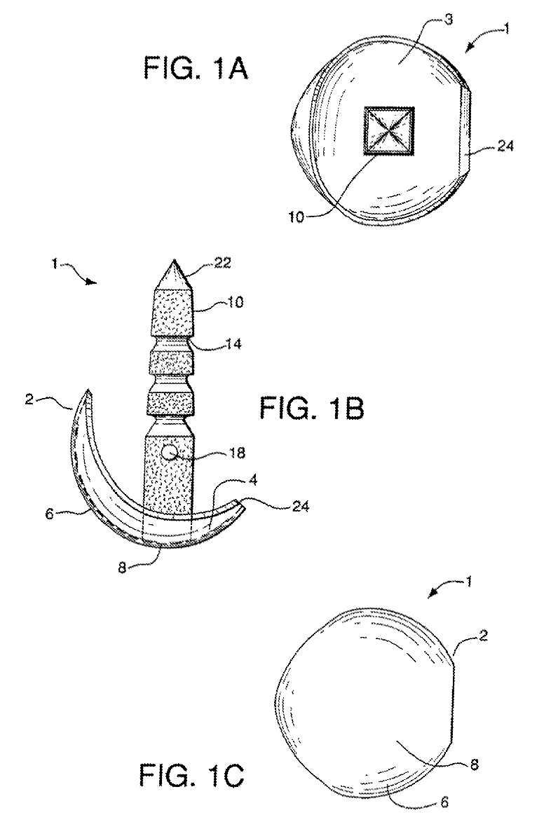

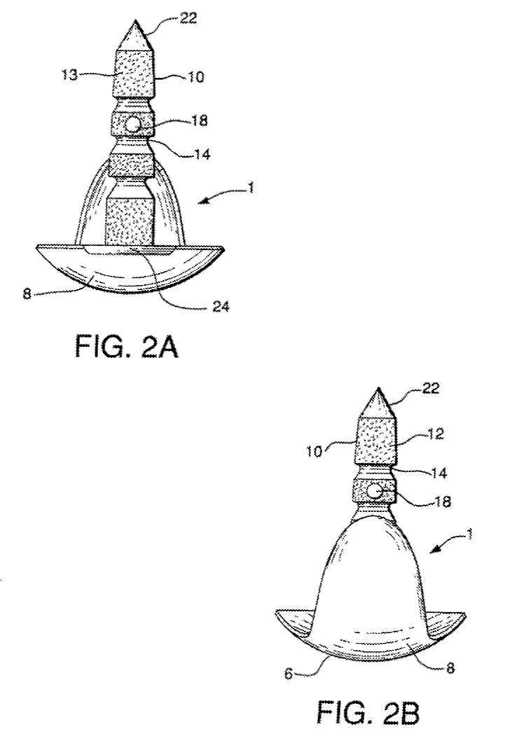

Referring to FIGS. 1-4, a metatarsal implant 1 has a head 2 that on the posterior side 3, defines a concave surface 4. The concave surface 4 is generally spherically shaped, having degrees of concavity that substantially conform to the shape of the distal joint end of a metatarsal bone, either naturally occurring or sculpted. Preferably, the degrees of concavity substantially conform to the shape of the distal joint end of the first metatarsal bone.

The joint end of a metatarsal bone may also be shaped into a standard form that conforms to the concave surface 4 of the implant. Such shaping may be necessary when the defect or disease of the metatarsal bone distorts the bone from the natural shape of the joint end of healthy metatarsal bone. Such shaping may be accomplished using a bone shaping tool such as a reamer and techniques generally known in the art.

The anterior face 6 defines a convex surface 8. The convex surface 8 is generally spherically shaped, having a degree of convexity that substantially conforms to the shape of the joint end of a phalanx. More preferably, the degree of convexity substantially conforms to the shape of the joint end of the first phalanx of the foot.

In one embodiment, the head of the implant maintains a substantially uniform degree of thickness throughout. In other embodiments, the head of the implant maintains varied degrees of thickness. Preferably, the thinnest areas of the head are along the edges of the head.

In one embodiment, the plantar edge 24 is truncated, truncated being defined as replacement of an edge by a plane, so as to avoid disturbance of the sesamoid apparatus by the implant head 2.

At least one stem 10 projects from the concave surface 4 of the implant away from the convex surface 8. Preferably, the stem projects generally normal to the concave surface of the implant. More preferably, the stem is positioned in the concave surface so as to allow the head 2 of the implant to project substantially over the stem 10.

The stem 10 may be present in a variety of shapes. In one embodiment, as seen in FIG. 1A, the stem has a substantially square cross-section. In others, the stem can have a substantially rectangular, triangular, or rounded cross-section (not shown). In one embodiment, the stem maintains a uniform diameter along the longitudinal axis of the stem. In other embodiments, the stem diameter progressively decreases along the longitudinal axis of the stem, the largest diameters being located near to the head of the implant.

In one embodiment, the stem 10 includes a plurality of serrations 14, serrations being defined as notches, barbs, or teeth, on one or more sides of the stem 10. The serrations 14 are preferably canted to provide for easy insertion of the stem 10 into the bone, yet resist removal or backing out of the stem from the cancellous portion of the metatarsal bone. The serrations 14 further provide a surface in which optional bone cement, or over time, bone tissue, will anchor the stem into the bone. Bone cement materials are known in the art and include materials such as those that are based on polymethylmethacrylate and calcium phosphate and others.

The stem 10 may also have a textured surface. The textured surface serves to encourage new bone growth to form on the implant to further secure the implant into the bone. Moreover, the stem may be coated with or comprise osteogenic or osteofacilitative material, likewise known in the art per se, to facilitate intimate bonding with the patient's own bone.

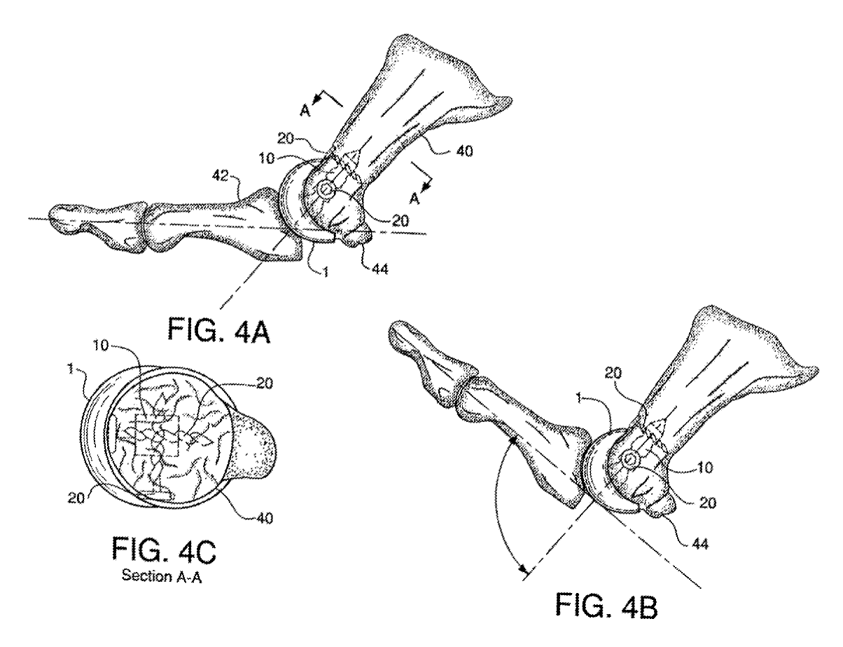

The stem 10 may also comprise at least one aperture 18 for accepting one or more fasteners (20), apertures being defined as channels or holes. The aperture 18 may be positioned in the stem such that one or more fasteners 20 may be inserted laterally into or through the stem (see FIGS. 4A, 4B, 4C). Alternatively, the apertures may be positioned in the stem such that the fasteners may be inserted from the dorsal side 12 of the stem to the plantar side 13 of the stem. Preferably, the stem 10 comprises a plurality of apertures such that fasteners may be inserted both laterally through the stem 10 and from dorsal (12) to plantar (13) sides of the stem 10.

When the implant has been inserted into the metatarsal bone, a template may be used to locate the stem apertures to facilitate fastener insertion. The template may comprise a plurality of markings indicating the distance from the anterior face 6 to an aperture 18. By positioning the template against the anterior surface of the implant, one may determine where inside the bone the apertures are located. Once the location of the apertures is determined, a hole may be drilled through the bone to allow for a fastener to be inserted through the bone, through the aperture, and through the stem.

In one embodiment, the fasteners 20 may be screws. In another embodiment, the fasteners may be self-locking screws. In yet another embodiment, the fasteners may be rods or pins, including structures which may be bioresorbable.

The stem 10 may also include a tip 22. Preferably the tip 22 may be in the shape of a trocar, a trocar being defined as a sharp, pyramidal shape. Preferably, the tip 22 may be a sharp, four-pointed, pyramidal shape. The tip 22 provides a surface capable of piercing or cutting tissue and bone to facilitate insertion of the stem into the bone.

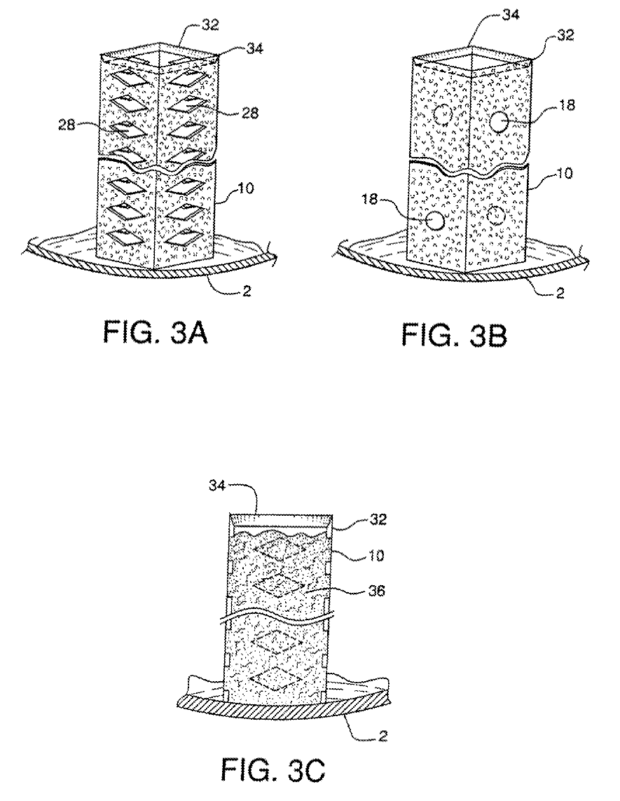

Referring to FIGS. 3A, 3B, and 3C, in another embodiment, the stem 10 may be hollow and may comprise a plurality of fenestrations 28, fenestrations being defined as openings. In other embodiments, the hollow stem may comprise a plurality of apertures 18. The hollow stem may further comprise an end portion 32, the end portion being distal to the head of the implant. The end portion may comprise a plurality of cutting surfaces 34, capable of cutting through tissue and bone so as to facilitate the implantation of tie stem into the bone. The hollow stem may be filled with osteogenic or osteostimulatory materials (36) in order to facilitate bone tissue growth to anchor the stem into the bone. Osteostimulatory materials are known in the art and include such materials as hydroxyapatite. Preferred among these, however is VITOSS®, sold by the Orthovita Company, Malvern, Pa. Such material may be mixed with blood and/or marrow harvested from the metatarsal site in order to facilitate bone regrowth. See e.g. U.S. Pat. No. 6,736,799.

The metatarsal implants of the present invention can be made of any suitable material. Preferably, the implant is made from medical-grade titanium alloy. Other acceptable materials include cobalt chromium, cobalt chromium molybdenum, stainless steel, aluminum, and ceramic. The metatarsal implants of the present invention may also be optionally coated.

Furthermore, it is preferred that a number of different sized implants, e.g., three or four sizes, be provided to account for different foot sizes.

The implant of the present invention may be implanted using techniques generally known to those skilled in the field. One method of implantation is provided by way of example.

A dorsal linear incision is initially made medial to the extensor hallucis longus tendon to expose and provide access to the metatarsal head and base, including the joint capsule and periosteum. Joint arthroplasty is then performed through the exposed first metatarsophalangeal joint to remove arthritic, diseased, and/or otherwise damaged bone.

A K-wire is centrally placed into the shaft of the first metatarsal bone and a conical reamer is placed over the K-wire. The first metatarsal head is then reamed to a rounded, contoured, anatomical shape. The reamer and K-wire are then removed and a sharp broach is placed centrally through the metatarsal head, down the metatarsal shaft, in preparation for the placement of the implant stem of the present invention. The broach is removed and implant sizers are placed into the metatarsal shaft. After an appropriate sizer has been determined, the sizer is removed and a likewise appropriately sized implant of the present invention is placed into the shaft of the first metatarsal bone. The implant is tamped to secure it in place.

Using a template guide, fasteners are threaded through the implant and secured. The wound can then be closed in a layered fashion. The procedure can also be performed using image intensification, as necessary.

Referring to FIGS. 4A and 4B, a metatarsal implant 1 constructed in accordance with the present invention is shown implanted into a first metatarsal bone. As shown in these figures, the implant 1 is implanted into the first metatarsal bone 40, abutting the joint end of the first phalanx of the toe 42. As shown in these figures, the anterior face 6 of the implant cooperates with the concave surface of the first phalanx 42, allowing a full range of motion about the metatarsophalangeal joint (FIG. 4B) without disturbing the sesamoid apparatus 44.

What is claimed:

1. A metatarsal implant comprising:
a substantially concavo-convex shaped body;
a concave portion of the shaped body conforming generally to the shape of an end of a metatarsal bone;
a convex surface of the body forming a bearing surface for a further bone of the foot;
the shaped body having at least one stem, the stem having a dorsal side and a plantar side, the stem being adapted for affixing the shaped body to the metatarsal bone.

2. The implant of claim 1 wherein the metatarsal bone is the first metatarsal bone.

3. The implant of claim 1 wherein the further bone is a phalanx.

4. The implant of claim 1 wherein the metatarsal bone is surgically shaped to conform to the concavity of the shaped body.

5. The implant of claim 1 wherein the stem projects perpendicularly from the concave portion of the shaped body away from the convex surface of the shaped body

6. The implant of claim 1 wherein the shaped body projects substantially over the dorsal side of the stem.

7. The implant of claim 1 wherein the stem has a square cross-section

8. The implant of claim 1 wherein the stem has a rectangular cross-section.

9. The implant of claim 1 wherein the stem has a triangular cross-section.

10. The implant of claim 1 wherein the stem has a curved cross-section.

11. The implant of claim 1 wherein the stem comprises sections, apertures, or both.

12. The implant of claim 1 wherein the stem further comprises a trocar tip.

13. The implant of claim 1 wherein the stem further comprises a sharp, three-sided tip.

14. The implant of claim 1 wherein the stem further comprises a sharp, four-sided tip.

15. The implant of claim 1 wherein the stem may accept at least one fastener.

16. The implant of claim 15 wherein the fastener is a screw.

17. The implant of claim 15 wherein the fastener is a self-locking screw.

18. The implant of claim 15 wherein the fastener is a rod.

19. The implant of claim 15 wherein the fastener is a pin.

20. The implant of claim 1 wherein the shaped body comprises cobalt chromium, cobalt chromium molybdenum, titanium, stainless steel, ceramic, or aluminum.

21. A metatarsal implant comprising:
a substantially concave-convex shaped body;
a concave portion of the shaped body conforming generally to the shape of an end of a metatarsal bone;
a convex surface of the body forming a bearing surface for a further bone of the foot;
the shaped body having at least one hollow stem, the hollow stem having a dorsal side and a plantar side, the stem being adapted for affixing the shaped body to the metatarsal bone.

22. The implant of claim 21 wherein the metatarsal bone is the first metatarsal bone.

23. The implant of claim 21 wherein the further bone is a phalanx.

24. The implant of claim 21 wherein the metatarsal bone is surgically shaped to conform to the concavity of the shaped body.

25. The implant of claim 21 wherein the hollow stem projects perpendicularly from the concave portion of the shaped body, away from the convex surface of the shaped body

26. The implant of claim 21 wherein the shaped body projects substantially over the dorsal side of the hollow stem.

27. The implant of claim 21 wherein the hollow stem has a square cross-section.

28. The implant of claim 21 wherein the hollow stem has a rectangular cross-section.

29. The implant of claim 21 wherein the hollow stem has a triangular cross-section.

30. The implant of claim 21 wherein the hollow stem has a curved cross-section.

31. The implant of claim 21 wherein the hollow stem comprises at least one of serrations, apertures, or fenestrations.

32. The implant of claim 21 wherein the hollow stem further comprises a distal portion.

33. The implant of claim 32 wherein the distal portion further comprises a plurality of cutting surfaces.

34. The implant of claim 21 wherein the stem is substantially filled with osteogenic material, osteostimulatory material, or both.

35. The implant of claim 21 wherein the shaped body comprises cobalt chromium, cobalt chromium molybdenum, titanium, stainless steel, ceramic, or aluminum.

36. A method for implanting a metatarsal implant, the method comprising the steps of providing a metatarsal implant comprising a substantially concavo-convex shaped body; a concave portion of the shaped body conforming generally to the shape of an end of a metatarsal bone; a convex surface of the body forming a bearing surface for a further bone of the foot; the shaped body having at least one stem; the stem being adapted for affixing the shaped body to the metatarsal bone;
conical reaming of the head of a metatarsal bone to form a contoured, anatomical shape;
placing and positioning of the stem and shaped body into the shaft of the metatarsal bone; and
securing the shaped body using fasteners inserted through the stem.
